(19) United States
(12) Patent Application Publication
(10) Pub. No.: US 2013/0101506 A1
(43) Pub. Date: Apr. 25, 2013

(54) COMPOSITIONS COMPRISING NUCLEIC ACID APTAMERS

(71) Applicant: Cassandra L. Smith, Boston, MA (US)

(72) Inventor: Cassandra L. Smith, Boston, MA (US)

(21) Appl. No.: 13/621,525

(22) Filed: Sep. 17, 2012

Related U.S. Application Data
(63) Continuation of application No. 11/495,252, filed on Jul. 28, 2006, now abandoned.
(60) Provisional application No. 60/703,274, filed on Jul. 28, 2005.

(51) Int. Cl. C12N 15/115; C07K 14/00; A61K 45/06; A61K 49/00; A61K 31/7088

(52) U.S. Cl. CPC C12N 15/115; A61K 49/00; A61K 31/7088; A61K 45/06; C07K 14/003
USPC 424/1.73; 536/23.1; 424/9.1; 424/9.35; 514/44 R; 530/300; 514/19.2; 424/93.1; 424/184.1; 424/94.1; 514/1.1; 424/600; 424/1.11

(57) ABSTRACT

Disclosed herein are aptamers that comprise a nucleic acid sequence that has a specific affinity for a target. These aptamers can be used as delivery vehicles to deliver specific agents to particular sites. Alternatively, targeted aptamers can also be used with detection techniques to determine the presence of absence of specific targets in heterogeneous backgrounds.

### CROSS-REFERENCE TO RELATED APPLICATIONS

This application is a continuation of co-pending U.S. patent application Ser. No. 11/495,252 filed on Jul. 28, 2006 and entitled COMPOSITIONS COMPRISING NUCLEIC ACID APTAMERS, which in turn claims priority to U.S. Provisional Patent Application No. 60/703,274 filed on Jul. 28, 2005, both of which are incorporated by reference herein in their entirety and for all purposes.

### STATEMENT OF FEDERALLY SPONSORED RESEARCH OR DEVELOPMENT

This invention was made in part with United States Government support under grant number DE-FG02-93ER61656, awarded by the United States Department of Energy and the U.S. Army Medical Research and Materiel Command (DAMD17-94-J-414) and the United States has certain rights in the invention.

### FIELD OF THE INVENTION

This invention relates to aptamers and to compositions comprising aptamers that have affinity for specific targets. The invention also relates to methods that are used to select aptamers and the employment of aptamers for diagnosis, treatment and detection of molecules indicative of a pathological process.

### BACKGROUND OF THE INVENTION

The search for molecules having high affinities and specificities for tumor components has been conducted for decades, and the usual final products are antibodies. The use of anti-tumor antibodies for in vivo detection and therapy have not been generally considered successful. Apart from poor target/non-target ratios (i.e. lack of specificity), murine antibodies often induce a human anti-mouse antibody response thereby preventing repeated administration of murine antibodies to humans. Even the use of chimeric or humanized antibodies does not provide for repeated administration since all antibodies have potential for inducing an anti-idiotype response. The pharmacokinetics of radiolabeled intact antibodies or their smaller fragments are unfavorable because both clear from the circulation and diffuse into tumor tissue slowly. Consequently, radioactivity accumulates non-specifically in various tissues, interfering with diagnosis and therapy. In addition, regulatory agencies often take a conservative view on the administration to patients of proteins that have been prepared in tissue culture or from mouse ascites.

Currently, there is intense interest in the use of nucleic acids as pharmaceuticals. At present, this interest has centered around antisense and siRNA applications in which oligonucleotides are used in cancer or other cells either to block transcription of genes within the nucleus or to block translation of or degrade mRNA within the cytoplasm. Most studies on the in vivo behavior of nucleic acids have been done primarily in connection with antisense applications where intracellular transport is of crucial importance and where the administered dosages are much larger than those contemplated here.

Natural phosphodiester-containing nucleic acids are rapidly degraded in vivo by nucleases so that their half-lives can be impracticably short for some radiopharmaceutical applications. In serum, oligonucleotides are predominantly degraded in a 3 exonucleolytic fashion. Thus, oligonucleotide stability in serum has been enhanced by covalently attaching a bulky group to the 3′ terminus or by modifying one or two of the nucleotides on this end. However, modifications solely at the 3′ end do not appear to completely stabilize oligonucleotides in vivo. More recent studies have focused on oligonucleotides containing a modified backbone. For instance, oligonucleotides with phosphorothioates and methyl phosphonate backbones were found to resist nuclease attack in vivo, hence, displaying greater in vivo stabilities.

One study using mice with a 20-base single stranded DNA (ssDNA) uniformly labelled with tritium, revealed that a monophosphorothioate oligonucleotide was much more stable in vivo than the equivalent phosphodiester. In serum, no degradation of the modified oligonucleotide occurred over a 24-hour period whereas an equivalent phosphodiester oligonucleotide was degraded within 30 minutes. Degradation occurred much more rapidly in liver and kidney tissue in the case of phosphodiester DNA. This investigation confirmed an earlier result showing rapid degradation of phosphodiester DNA using uniformly 35S-labeled oligonucleotides. Phosphodiester DNA administered intraperitoneally to mice failed to invoke any toxic response. The label appeared in most tissues within 48 hours after intravenous or intraperitoneal, administration of 30 mg/kg of body weight. Only about 30% of the label was secreted in urine during the first day, in the first 6 hours post administration primarily as intact DNA. In circulation, oligonucleotides remained intact for over 24 hours.

Early human patient trials appear to show similar pharmacokinetic properties to those seen in mice, rats, rabbits, and monkeys. In all cases, 30-40% of the phosphorothioate oligonucleotides were excreted in urine within 24 hours of administration. Remaining oligonucleotides accumulate, mostly in the liver where they are slowly cleared, and to a lesser extent in the spleen.

No toxicity problems have been associated with the use of antisense phosphodiester oligonucleotides in animals. In one study, dosages up to 60 mg/kg of body weight of a 20-base phosphodiester DNA administered intraperitoneally to mice failed to evoke any toxic response. Although substitution of a phosphorothiolate led to some discomfort at the highest dosages (160 mg/kg), all animals recovered within 24 hours. The modified oligonucleotides appeared to be non-toxic at doses of equal to or lower than 40 mg/kg of body weight. In rats, no mortality was observed at the highest dosage (150 mg/kg body weight).

Currently, sparse information is available on the toxicity of oligonucleotides in humans. At the highest dosages, up to 4-5 gm/patient, apparently needed for antisense therapy, there are indications that oligonucleotides stimulate the release of tumor necrosis factor. At the lower dosages, there is no evidence of toxicity.

Aptamers are DNA and RNA oligonucleotides due to their secondary and tertiary structures that bind with high affinity and selectivity to a target molecule. In other words, the structure of a particular aptamer allows it to bind to a specific molecule, such as a tumor-associated antigen. While alternative approaches to identifying receptor binding to ligands exists, the aptamer approach has the advantage of allowing large numbers of ligands to be generated by standard method and tested for binding against a number of targets. Aptamers are selected in vitro using libraries of synthesized nucleic acids. The libraries are screened by affinity chromatography methods to select those molecules that have high affinity for a specific target, and low affinity for other, competing targets.

The aptamer approach was first used by Ellington and Szostak (A D Elllington, J S Szostak, Nature 346; 818-822, 1990) who isolated singe stranded RNAs (ssRNA) aptamers that bound to six dyes of relatively low molecular weight. In this pioneering work, a 100-base DNA oligonucleotide library was synthesized en mass using standard phosphoramidite chemistry. Each member of the library had constant sequences on both ends to allow for PCR amplification and in vitro transcription. The variable middle portion of the oligonucleotides was synthesized by using an equimolar mixture of the four bases at each of the positions. A pool of RNAs of this size should have a large number of different secondary and tertiary structures. A 100 microgram samples of the library should consist of approximately 1015 unique RNAs. The constant primer binding sequences by about 20-fold with PCR in order to provide enough material for analysis. These products were converted to RNAs using T7 RNA polymerase. Six dye separately conjugated to a matrix were used independently as an affinity surface for the capture of aptamers with high binding affinities. This initial investigation used RNA rather than DNA because DNA was not thought to possess the structural diversity of RNA. However, subsequent studies established the use of single stranded DNAs (ssDNAs) as aptamers.

The aptamer approach was used to identify a class of short, ssDNAs that bind to, and inhibit, thrombin (L C Griffin, J L Toole, L L K Leung. Gene 137: 25-32, 1993). A pool of 96-base DNAs containing a 60 base random sequence flanked by 18 base constant sequences (to facilitate PCR amplification) was chemical synthesized. PCR amplification was done using a biotinylated 5 end primer to facilitate purification. The initial library contained more than 1013 unique molecules. Concanavaline A (ConA) binding aptamers were removed by passage over a ConA-agarose affinity column. Then, thrombin-binding-aptamers were selectively captured on thrombin immobilized on ConA. The column was washed to remove all unbound and weakly bound DNA before the DNA of interest was eluted with an excess of ConA ligand (alpha-methyl mannose). The fractions with thrombin were pooled and the DNA in these fractions was isolated and amplified 100-fold by PCR. This selection cycle was repeated a total of five times. Only a small fraction of the original DNA pool was ultimately recovered after five cycles. The isolated aptamers bound prothrombin as well as thrombin and had no affinity for plasminogen activator, albumin, kallilkrein, trypsin and chymotrypsin. DNA sequence analysis revealed a striking conservation in a 14-17 base regions containing a tandem duplicated hexamer consensus sequence. The highest affinity aptamer for thrombin had a binding constant of about 108 M. This affinity is comparable to many antigen-antibody reactions. This work demonstrated that DNA, like RNA, was capable of forming specific aptamers.

Today, the aptamer concept has been successfully applied to developing nucleic acid ligands that bind small molecules and a variety of proteins (summarized at website aptamer.icmb.utexas.edu/news.html) with affinities that range up to 10-13 M. Aptamers have been selected against small inorganic molecules such as zinc, metabolites such as cyclic AMP and vitamin B12, proteins from many different structural and functional classes (kinases, cytokines, proteases, transcription factors, to name but a few), supramolecular structures such as viruses, cells, tissues, and organisms (L. Gold et al., Ann. Rev. Biochem 64: 763-713, 1995,: S. E. Osborne and A. D. Ellington. Chem. Rev, 97: 349-370, 1997; M. Famulok and G. Mayer, Curr. Topics Microbiol. Immunol. 243: 1232-436; 1999; T. Hermann and D. J. Patel. Science 287: 820-825, 2000). In general, aptamers bind their cognate targets with high affinities (KD values in the nanomolar range) and specificities (aptamers can discriminate between small organics on the basis of single methyl or hydroxyl groups, and between proteins on the basis of single amino acid changes).

### SUMMARY OF THE INVENTION

The present invention relates to aptamers, to compositions comprising aptamers that have affinity for specific targets and to methods to isolate aptamers. The invention also relates to methods that employ aptamers for detection, diagnosis and treatment of molecules and/or cells indicative of a pathological process. These methods may be in vitro or in vivo.

Aptamers are DNA and RNA oligonucleotides that have secondary and tertiary structures that facilitate high affinity binding with a target molecule. In other words, the structure of a particular aptamer allows it to bind in an analogous fashion to an antibody binding to its target.

One embodiment of the present invention pertains to an aptamer comprising of a nucleic acid sequence coupled to an agent wherein the aptamer nucleic acid sequence is specificity for a predetermined target. The aptamer sequence can comprise one or more constant and variable regions that can be used to facilitate amplification, replication or cleavage of the aptamer. The aptamer sequence can be comprise of a sequence of DNA, RNA, or PNA (peptide nucleic acid), with natural or non-naturally occurring bases having a phosphodiester, phosphorothioate or methylene phosphorothioate chemical backbone, peptide or other modification. Coupling of an agent to an aptamer is dictated by the nature of the agent, but typically involves well-known coupling techniques including chemical coupling utilizing chelators, for example, DPTA or SHNH, or by chemical conjugation or non-covalent attachment. The agent can be another sequence, (e.g., antisense, siRNA) or other therapeutic agent useful for the detection, diagnosis or treatment of a disorder. Useful agents include, for example, radioisotopes, stable isotopes, pharmaceutical compounds, paramagnetic labels for MRI imaging, and fluorescent labels, macromolecules and other chemical and biological substances. One or more agents can be coupled to the aptamer sequence via chemical chelators or other suitable means of covalent or non-covalent attachment.

Another embodiment of the instant invention relates to the preparation of nucleic acid aptamers. Libraries comprising a plurality of nucleic acids may contain the same or different types of nucleic acids. Library variation can be due to nucleotide sequence variation, variation in the sequence or size or variation in the 3-dimensional structures. Further, libraries can be fixed to a solid support or free in solution.

Another embodiment of the invention is directed to methods for treating a disorder comprising the administration of an effective amount of an aptameric composition. Aptamers to be administered comprise an agent that functions as a therapeutic agent designed to treat the disorder. The agent can be a pharmaceutical compound, an antibody, an enzyme, a functional nucleic acid and alike. The target includes, but is not limited to, a diseased cell (such as a tumor cell), cell-surface antigen, diseased tissue (such as a tumor cell), and alike.

Still another embodiment of the invention is directed to methods for treating a neoplastic disorder comprising administering to a subject in need thereof a pharmaceutical composition comprising a predetermined aptameric, wherein the aptamer is coupled to a therapeutic agent, for example, a radioisotope containing moiety. This radioisotope moiety can be a nuclide used for imaging or treatment. In one aspect, aptamers administered are targeted to a site that is characteristic of the disorder. Such target sites include tumor-associated antigens, infections and other foreign antigens, cell-surface antigens, other disease-specific enzymes and alike.

Yet another embodiment of the invention is directed to methods for the detection of a target. The aptamers of the present invention have a predetermined affinity to one or more targets, administration of a composition comprising one or more aptamers covalently or non-covalently associated or non-associated will deliver the aptameric composition to the target. Detectable agents coupled to an aptamer allow for detection of the presence or the absence of the target in vitro or in vivo. In vitro assays may be homogenous (in solution), or heterogeneous (use of an immobilized component, either the aptamer or the target). In vivo assays can be within a surgical or other field of scrutiny and may encompass the entire body.

### BRIEF DESCRIPTION OF THE DRAWINGS

FIG. 1 is a diagram of the double SELEX protocol for isolation of anti-CEA aptamers.

FIG. 2 is an autoradiogram showing library complexity by following the appearance of specific restriction fragments from end-labeled DNA pools.

FIG. 3 is a sequence alignment of first-generation anti-CEA aptamers and consensus sequences.

FIG. 4A is a graph showing binding and release of clone26bases1-50 to CEA in solution.

FIG. 4B is a graph showing binding as a function of CEA concentration.

FIG. 4C is a graph showing binding as a function of CEA concentration in the linear range.

FIG. 5 is a schematic of multivalent selection methods.

FIG. 6A is a schematic of a bivalent antibody.

FIG. 6B is a schematic of single-stranded aptamers.

FIG. 6C is a schematic of double-stranded aptamers.

### DETAILED DESCRIPTION

The present invention relates to aptamers. Methods for isolating specific aptamers are disclosed. Also, methods of detecting predetermine targets employing aptamers specific for the targets are also disclosed herein. Other methods, including methods for detecting, diagnosing, treating or preventing a disorder using aptamers are also described herein.

Aptamers are DNA and RNA oligonucleotides that have secondary and tertiary structures that have high affinity and specific binding to a target molecule. In other words, the structure of a particular aptamer allows it to bind in an analogous fashion to an antibody binding to its target.

Aptamers can be generated by using molecule capture technologies. (A. D. Ellington and J. W. Szostak. Nature 346: 818-822, 1990, C. Tuerk and L. Gold. Science 249: 505-510, 1990, the teachings of which are incorporated herein by reference). A large number of nucleic acid ligands, including both DNA and RNA ligands, have been developed that bind small molecules as well as a variety of macromolecules (see website aptamer.icmb.utexas.edu/news.html). A major advantage of the aptamer approach is the ease with which large numbers of random nucleotide sequences can be prepared, amplified and tested. As a consequence, proteins and smaller molecules with no known affinity for oligonucleotide binding have been shown to be bound by aptamers when the latter have been property selected. Further, aptamers are fairly non-toxic and non-immunogenic (H. U. Hormann and H. U. Goringer, 1999. Nucleic Acids Res. 27: 2006-2014, 1999, the teaching of which is incorporated herein by reference) and can be safely administered at fairly high doses, higher than is necessary for most applications, for instance, 7 mg/kg/day (K. Pietras. Cancer Res. 61: 2929-2934, 200, the teaching of which is incorporated herein by reference) used for the anti-PDGF aptamer in rats to inhibit human tumor xenograft growth. Aptamers are also safely and rapidly eliminated from a biological system without the creation of harmful or toxic by-products, (See, Drolet. Pharmaceutical Res. 17:1503-1510, 2000 for the anti-VEGF aptamer, the teaching of which is incorporated herein by reference.)

It has been discovered that aptamers can be utilized as novel targeting devices for the delivery of molecular agents to specific target sites. These molecular devices are useful for the detection and treatment of disorders typically by targeting molecules like proteins, both in vivo and in vitro that are associated with a particular physiological event. For example, many tumors have associated tumor-specific antigens or ligands associated with them (J. Cohen, Sci. 262:841-43, 1993, the teaching of which is incorporated herein by reference). Tumor-binding aptamers can be employed directly or indirectly to aid tumor targeting. By coupling aptamers with therapeutic agents, tumor cells can be specifically targeted for treatment or destruction. Further, novel tumor-specific molecules can be identified by screening oligonucleotide aptamer libraries with whole tumor cells. Alternatively, a reverse selection using immobilized aptamers can provide a method for identifying tumor specific targets. Upon identification, aptamers of the invention can be prepared and utilized for imaging, treatment as well as other applications.

One embodiment of the invention is directed to a targeted aptamer comprised of a nucleic acid sequence coupled to an agent wherein the sequence has specificity for a particular target. Aptamers, although nucleic acid sequences, can be distinguished in that they have a specific affinity for a target and have different sequences. Many different types of aptamers have been identified and characterised (at website aptamer.icmb.utexas.edu/news.html) including, for example, aptamers that bind small molecules such as organic dyes, antibiotics, the alkaloid theophylline (R. D. Jenison et al., Sci. 263:1425-29, 1994), as well as a variety of macromolecules including proteins such as thrombin, bacteriophage T4 DNA polymerase (C. Tuerk et al., Sci. 249:505-10, 1990, the teaching of which is incorporated herein by reference), and the bacteriophage R17 coat protein (CA. Stein et al., Sci. 261:1004-11, 1993, the teaching of which is incorporated herein by reference), nucleolin (Dapic et al., Biochem 41: 3676-3685, 2002, the teaching of which is incorporated herein by reference), PSA (Lupold et al., Cancer Research 61: 4029-4033, 2002, the teaching of which is incorporated herein by reference), and VEGF (Ruckman et al., J. Biol. Chem 273: 20556-20567, 1998, the teaching of which is incorporated herein by reference).

Generally, aptamers are identified and isolated from pools of nucleic acid sequences using techniques that are known to those of ordinary skill. For example, aptamers can be selected by incubation with a target molecule. Oligonucleotides that bind to the target can be selected, amplified (e.g. by polymerase chain reaction (PCR) techniques), and further purified using, for example, an affinity column composed of target molecules. These techniques can be applied to nucleic acids of lengths from about 10 nucleotides to about 200 nucleotides or more. In one aspect, the aptameric oligonucleotide is between about 20 and 200 nucleotides in length. In another aspect, the aptameric oligonucleotide is less than 100 nucleotides in length.

The aptamer sequence comprises a sequence of DNA, RNA or PNA, and can contain naturally or non-naturally occurring bases. The natural bases are adenine (A), guanine (G), cytosine (C), thymine (T), inosine (I), and uracil (U). Some of the non-naturally occurring bases include, for example, methylinosine, dihydrouridine, methylguanosine, thiouridine and many others well known to those of ordinary skill in the art. PNA bases can include natural or non natural bases attached to an amide (peptide-like backbone). The backbone of nucleic acid sequence can be an amide such as PNA, or a phosphodiester such as in DNA or RNA, a thiophosphodiester, a phosphorothioate, a methylene phosphorothioate or a modification of these chemical structures.

The nucleic acid sequence of the aptamer can comprise only the target-binding sequences. The aptamer can comprise a constant and a variable region. In one aspect, the target binding sequence is in the variable portion. In another aspect, the target-binding sequence is in the constant region or in both the variable and constant regions. Typically, constant region sequences can be used to facilitate binding, amplification, replication or cleavage of the sequence. Constant regions are typically at either or both the 5′ and 3° termini of the sequence to facilitate cloning, restriction endonuclease digestion, the binding of oligonucleotide primers for PCR or other amplification techniques. The sequence can be entirely unique or constructed using stretches of homopolymers or repeated sequences of bases as necessary to form the aptamer structure.

Aptamers of the present invention can be coupled to agents that are delivered to the target or target site for detection, imaging, or other diagnostic purposes, for therapeutic or prophylactic treatment of diseases and disorders, or for the creation of molecular structures at the target site. Agents can be for the treatment of a disorder (therapeutic or prophylactic) include cells, nanoparticles, hormones, vaccines, haptens, toxins, enzymes, immune system modulators, anti-oxidants, vitamins, functional agents of the hematopoietic system, proteins, such as streptavidin or avidin or mutations thereof, metals and other inorganic substances, virus particles, antigens such as amino acids, peptides, saccharides and polysaccharides, receptors, radioisotopes and radionuclides such as is 93P, 95mTc, 99Tm, 186Re, 188Re, 189Re, 111In, (also useful as imaging agents) 14C, 32P, 3H, 60C, 125I, 35S, 65Zn, 124I and 226 Ra, and stable isotopes such as 3He, 6Li, 10B, 113Cd, 135Xe, 149Sm, 151Eu, 155Gd, 174Hf, 199Hg, 235U, 241Pu, and 242Am, paramagnetic and fluorescent labels, pharmaceutical compounds or other macromolecules.

Pharmaceutical compounds that can be coupled to aptamers include, for example, conventional chemotherapeutic agents such as cyclophosphamide, alkylating agents, purine and pyrimidine analogs such as mercaptopurine, vinca and vinca-like alkaloids, etoposides, or etoposide-like drugs, antibiotics such as deoxyrubocin and belomycin, corticosteroids, mutagens such as the nitroureas, antimetabolites including methotrexate, platinum based cytotoxic drugs, hormonal antagonists such as anti-insulin and antiandrogen, antiestrogens such as tamoxifen and other agents such as doxorubicin, L-asparaginase, dacarbazine (DTIC), amsacrine (m-AMSA), procarbazine, hexamethylmelamine, and mitoxanthrone.

Macromolecules that can be coupled to an aptamer include mitogens and cytokines, growth factors such as B cell growth factor (BCGF), fibroblast-derived growth factor (FDGF), granulocyte/macrophage colony stimulating factor (GM-CSF), granulocyte colony stimulating factor (GCSF), macrophage colony stimulating factor (M-CSF), epidermal growth factor (EGF), platelet-derived growth factor (PDGF), nerve growth factor (NGF), stem cell factor (SCF), and transforming growth factor (TGF). These growth factors plus other composition can further stimulate cellular differentiation and/or the expression of certain MHC antigens or tumor specific antigens. In a similar fashion, other agents such as differentiating agents can be useful to prevent or treat a neoplastic disorder. Other differentiating agents include B cell differentiating factor (BCDF), erythropoietin (EPO), steel factor, aetivin, inhibin, the bone morphogenic proteins (BMPs), retinoic acid or retinoic acid derivatives such as retinol, the prostaglandins, and TPA.

Alternatively, other cytokines and related antigens, coupled with targeted aptamers, can also be useful to treat or prevent disorders such as neoplasia. Potentially useful cytokines include tumor necrosis factor (TNF), the interleukins (IL-1, IL-2, IL-3, etc), the interferon proteins IFN) IFN-α, INF-β, and IFN-M, hormones including glucocorticoid hormones, cytosine arabinoside, and anti-virals such as acyclovir and gancyclovir.

Aptamers of the present invention are targeted to specific targets that can be determined empirically be preselected or identified by testing. Desirable targets include microorganisms, enzymes, pharmaceutical compounds such as drugs (including illegal drugs), antigens, cells of the immune system or other systems, proteins and other macromolecules such as toxins, immune system modulators, and secondary and tertiary nucleic acid structures.

Target sites include diseased or normal cells, particles whose presence of absence may be desirable, microorganisms such as virus particles, bacteria, parasites, fungus and their respective characteristic antigen(s). Target sites can also be a site-specific phenomenon such as topological features attendant to a neoplastic cell or particle surface.

Coupling of an aptamer to an agent can be performed using well-known methods, including chemical and biological techniques. Covalent bonds or non-covalent interaction can be created depending upon the type of interaction desired (e.g., C.-P. D. Tu et al., Gene 10:177-83, 1980; A. S. Boutorine et al., Anal. Biochem. Bioconj. Chem. 1:350-56, 1990; S. L. Commerford Biochem, 10:1993-99, 1971; D. J. Hnatowich et al., J. Nucl. Med. 36:2306-14, 1995, the entire teachings of which are incorporated herein by reference.) For example, covalent bonds that can be formed include those formed from chemical conjugation reactions, bonds formed from coupling utilizing chelators, or bonds formed from phosphodiester linkages. Non-covalent bonds include molecular interactions such as those between streptavidin and biotin, hydrogen-bonding and other forms of ionic interaction. Chelators that can be used to facilitate coupling include, for example, DTPA, SHNH and multidentate chelators such as N2S2 and N3S (A. R. Fritzberg et al., J. Nucl. Med. 23:592-98, 1982, the entire teaching of which is incorporated herein by reference.) and the tetradentate (four-fold) chelator moiety MAG-3 (N—[N—[N-[(benzoylthio)acetyl]glycyl]glycyl]glycine (R. W. Weber et al., Bioconjug. Chem. 1:431-37, 1990, the entire teaching of which is incorporated herein by reference). These types of chelation reactions are encouraged by the coupling of another chemical moiety containing, for example, a reactive amino or carboxyl group to the sequence. In one aspect, the 5′ or 3′ terminus of the sequences include aminohexyloligonucleotide (AHON) that conjugates the aminohexyl moiety to diethylanatriaminepantancetateisothiocynate (DTPAI) (M. K. Dewajee et al., Advances in Gene Technology: The Molecular Biology of Human Genetic Disease p. 76, the entire teaching of which is incorporated herein by reference). Aptamers may also be bound to the cell surface (e.g. red blood cell surface) or to a nanoparticle to guide such structures to a specific location in vitro or in vivo.

Another embodiment of the instant invention is directed to libraries of aptamers comprising a plurality of aptameric oligonucleotides that can be the same or different. Libraries can be fixed to a solid support such as a surface (e.g. slide, well, tube, sheet, membrane, bead, nanoparticle), a silicone or other form of chip or microchip, or free in solution. Library variability can be due to sequence variation or to variations in the size of the variable region. Libraries can be used for identifying specific aptamers or for screening for aptamers of a particular conformation. In addition, libraries can be fixed to a solid support or free in solution as desired.

Aptamers are selected using an approach called the selective evolution of ligands by exponential enrichment (SELEX) process (Ellington et al., 1990; Tuerk et al., 1990, the teaching of which is incorporated herein by reference). SELEX is a method for screening very large combinatorial libraries of oligonucleotides by a repetitive process of in vitro selection and amplification.

In SELEX, a random sequence oligonucleotide library is incubated with a target. The steps involved in selection of an aptamer are:
(A) The SELEX process begins with a random sequence library obtained from chemical synthesis of DNA. A typical library comprises a large number of different targeted aptamers, typically greater than 1014 different aptamers, in another aspect greater than about 106 different aptamers, and in another aspect greater than about 108 different aptamers, or more. The chemical synthesis of an initial library containing conserved primer regions flanking a random sequence that is PCR amplified to increase the number of distinct sequences. A modified or unmodified DNA library is synthesized at the μM scale. The synthetic molecules generally have 20-100 bases of variable sequence flanked by conserved primer regions for amplification. Interactions between the primer ends are discouraged by careful sequence selection. Library size and therefore complexity is limited by the scale of current solid phase synthetic technology to 1 μmole and results in ˜1017 different molecules. Complete library coverage can be achieved with 25 random bases 425=1.1×1015 assuming all varying sequences are synthesized with no duplication. The initial PCR creates ˜10 copies of each unique oligonucleotide.
(B) The library is incubated with the immobilized target molecule. During this step, a very small fraction of individual sequences interacts with the target. These sequences bound to the target may be separated from the rest of the library by means of any one of many physical separation techniques. Typically, affinity chromatography or capture on nitrocellulose filters are used,
(C) The unbound sequences are discarded.
(D) The bound sequences are isolated and PCR amplified and prepared for another round of SELEX. After each round the library is enriched for sequences with an affinity towards the chosen target and the numbers of low affinity sequences are reduced.

The enrichment efficiency of high-affinity binders is governed by the stringency of selection at each round. The progress of the enrichment of high-affinity binders can be determined by analysis of the amount of radiolabeled oligonucleotide library binding to the target and by indirect-end labelling experiments to measure the complexity of the library composition. Once affinity saturation is achieved after several rounds of selection/amplification, the enriched sequence library is cloned and sequenced to obtain the sequence information of individual members and to determine whether a conserved sequence motif is present in the sequences.

Another embodiment of the invention is directed to methods for detecting or treating a disorder comprising the administration of an effective amount of a composition comprising one or more aptamers coupled to one or more of therapeutic agents. In one aspect, compositions comprise pharmaceutically acceptable carriers such as water, oils, lipids, starches, cellulose, saccharide and polysaccharide, glycerol, collagen and combinations of these carriers. Compositions can further comprise reagents that stabilize and preserve the active ingredients.

Compositions comprising aptamers can be administered to a subject such as humans or other mammals. Direct administration of a composition can be oral, parenteral, pulmonary absorption or topical application. Parenteral administration is preferred by intravenous injection, subcutaneous injection, intramuscular injection, intra-arterial injection, intrathecal injection, intra-peritoneal injection or other administration to one or more specific sites. Injectable forms of administration are sometimes preferred for maximal effect in, for example bone marrow.

When long-term administration by injection is necessary, venous access devices such as mediports, in-dwelling catheters, or automatic pumping mechanisms are also preferred wherein direct and immediate access is provided to the arteries in and around the heart and other major organs and organ systems.

Another effective method of administering aptamer-containing compositions is by direct contact with, for example, bone marrow through an incision or some other artificial opening into the body. Compositions can also be administered to the nasal passages as a spray. Arteries of the nasal area provide a rapid and efficient access to the bloodstream and immediate access to the pulmonary system. Access to the gastrointestinal tract, which can also rapidly introduce substances to the bloodstream, can be gained using oral, enema, or injectable forms of administration. Aptamer compositions can be administered as a bolus injection or spray, or administered sequentially over time (episodically) such as every two, four, six or eight hours, ever day (QD) or every other day (QM), or over longer periods of time such as weeks to months. Compositions can also be administered in a timed-release fashion such as by using slow-release resins and other timed or delayed release materials and devices.

Pharmaceutical compositions of the present invention comprise a therapeutically effective amount of one or more aptamers. Therapeutically effective amounts can be determined from empirical testing and from evaluating published information regarding the administration of antisense or other nucleic acid therapeutics nucleic acids. As discussed above, these and other studies have indicated that nucleic acids are safe and non-toxic at effective doses. Based on these studies, a therapeutically effective amount is between 10 mg/kg patient body weight to about 50 mg/kg, in another aspect between about 0.1 mg/kg patient body weight to about 25 mg/kg patient body weight, and in still another aspect between at 1.0 mg/kg patient body weight to about 10 mg/kg body weight. The therapeutically effective dose can also be determined by those of ordinary skill from the available literature on effective dose of each component of the composition.

Aptamers can be coupled to agents that are useful to treat or detect a disorder. The target of the composition can be a diseased cell or cell-surface antigen, or diseased tissue and is preferably a tumor cell. A wide variety of disorders can be treated including any disease or malady that could be characterised as a neoplasm, a tumor, a malignancy, a cancer or a disease which results in a relatively autonomous growth of cells. Neoplastic disorders prophylactically or therapeutically treatable with compositions of the invention include small cell lung cancers and other lung cancers, rhabdomyosarcomas, choriocarcinomas, glioblastoma multiforme (brain tumors), bowel and gastric carcinomas, leukemias, ovarian cancers, prostate cancers, osteosarcomas, or cancers which have metastasized. Diseases of the immune system which are treatable by these compositions include the non-Hodgkin's lymphomas including the follicular lymphomas. Burkitt's lymphoma, adult T-cell leukemias and lymphomas, hairy-cell leukemia, acute myelogenous, lymphoblastic or other leukemias, chronic myelogenous leukemias, myelodysplastic syndromes, breast cell carcinomas, melanomas and hematologic melanomas, ovarian cancers, pancreatic cancers, liver cancers, stomach cancers, colon cancers, squamous cell carcinomas, neurofibromas, testicular cell carcinomas and adenocarcinomas. Additional diseases treatable by the compositions include virally-induced cancers wherein the viral agent is EBV, HPV, HTLV-1, or HBV. Such a wide variety of disorders can be treated because aptamers are such a general means of delivery.

Another embodiment of the invention is directed to a method for imaging or treating a neoplastic disorder comprising administering to a subject an effective amount of a pharmaceutical composition comprising one or more aptamers. These aptamers are comprised of a nucleic acid sequence (e.g. DNA, RNA, PNA) to which is coupled a therapeutic or imaging agent such as an isotope. The isotope may be a metallic radionuclide for imaging purposes or a radioisotope for therapeutic use. Nucleic acids may be labelled with those of ordinary skill with 32P or 33P with conventional recombinant methods for instance using kinase or DNA polymerase. Aptamers may be labelled by conventional methods with fluorescein or other labels that allow visualization in a surgical or other fields containing neoplastic cells or other non-native or native cells.

A number of methods available have been developed for labelling with radionuclides for imaging and therapeutic uses including those described by D. J. Hnatowich et al, J. Nuclear Medicine 36: 2306-2314, 1995; C. K. Younes, R. Boisgard, B. Tavitan, Curr. Phar. Des 8: 1451-1466, 2002; F. Dolle et al., J. label Compounds Radiophar. 39: 319-330; B. Tavitian. Nat. Med. 4: 467-471, 1998; Y. M. Zhang et al., Eur. J. Nucl. Med. 27; 1700-1707, 2000; I. G. Panyutin et al., Q. J. Nucl. Med. 44: 256-267, 2000 the entire teachings of which are incorporated herein by reference).

For example, there are numerous methods for labelling oligonucleotides with technetium-99m (99mTc) or indium-111 (111In), both of which have been shown to have imaging applications. For instance, aptamers coupled to a radionuclide was synthesized with a biotin moiety on either the 3′ or 5′ using end and a primary amino group at the opposite end. The biotin was used to immobilize the oligonucleotide on streptavidin-conjugated magnetic beads while the amino group was derivatized to permit radiolabeling. Conjugation with diethylene triaminepentaacetic acid (DPTA) permits stable labelling with 111In while conjugation with hydrazinonicotinamide (SHNH) permitted stable labelling with 99 mTc. Thus, there are numerous methods for stably labelling any amino-derivatized oligonucleotides with radionuclides.

Labelled oligonucleotides can be used in radioimmuno targeting of tumor cells in vivo with antibodies or other targeting reagents. Streptavidin and avidin are tetramers that can bind 4 biotins or biotinylated molecules. Recombinant forms of streptavidin are used as a linker to bind biotinylated antibody, DNA and other molecules, cells, surfaces, particles etc (T. Sano et al., Proc. Nat. Acad. Sci. 87:1142046, 1990, the teaching of which is incorporated herein by reference). The streptavidin-biotin system provides for a two-step, in vivo, targeting protocol where radiolabeled biotinylated oligonucleotides are targeted to an antibody-streptavidin complex previously bound to tumor cells (or vice versa). Further, other experiments are exploring the use of sets of complementary oligonucleotides, which are be bound to streptavidin, and are capable of forming super complexes as non-enzymatic amplification tools to create multiple DNA targets on tumor cells. Other experiments have explored the use of a chimeric streptavidin-metallothionein fusion for radioisotope delivery. All of these experiments involve the use of antibodies for tumor targeting to which the aptamer approach can be applied. In a similar approach, the targeting may involve the use of two or more targeting reagents composed of nucleic acids (or other composition), and involve the formation of double stranded DNA or RNA.

Another embodiment of the invention is directed to methods for the detection of a target. Targeted aptamers are administered to a patient or other body that may contain the target. Detectable agents coupled to the aptamer allow for identification of the target. Detectable agents include, for example, chemical moieties that possess a specifically identifiable physical or chemical properties that can be distinguished such as, for example, fluorescence, phosphorescence and luminescence including electroluminescence, chemiluminescence and bioluminescence. In addition, reactive derivatives of dansyl, coumarins, rhodamine and fluorescein are all inherently fluorescent when excited with light of a specific wavelength and can be specifically bound or attached to nucleic acids. Coumarin has a high fluorescent quantum yield, higher than even a dansyl moiety, and facilitates detection where very low levels of target that are being sought.

It may also be useful to combine certain detectable moieties to facilitate detection or isolation. Other detectable agents include, for example, metals, radioactive, magnetic and electro-magnetic substances, and enzymes. By administering a plurality of targeted aptamers, even very rare targets can be successfully detected.

Another embodiment of the invention is directed to a method for inactivating a biological target comprising the steps of administering to a patient targeted aptamer comprising a nucleic acid sequence that inactivates or blocks function directly or is coupled to an inactivating agent. Inactivating agents include, for example, isotopes, toxins, or photodynamic or photosensitive agent. In such methods, the biological target may be a virus, a bacterium, a fungal organism, a prion, a nucleic acid, a cell, a parasite, an infectious agent, a hormone, an immune-modulator, an enzyme, a toxin, or a protein. Upon placing the agent in close proximity to or in contact with the target, the target can be destroyed or inactivated.

An embodiment of the invention is directed to a method for preparing a targeted aptamer comprising the steps of preparing a nucleic acid sequence that is specific for a target and coupling that sequence to an agent. The agent may have therapeutic, imaging, diagnostic, or targeting applications. The sequence may be coupled to the therapeutic agent via spacer or a linker. Methods may further comprise the step of purifying or even identifying the targeted aptamer by, for example, affinity chromatography using the target as the affinity reagent.

Another embodiment of the invention is directed to kits that contain targeted aptamers of the invention. These aptamers are coupled to agents that are useful for the detection of target in a biological or other sample. Biological samples may be obtained from, blood, plasma, serum, or other bodily fluids or tissues or be composed of a medical or surgical fields. By coupling the aptamer to a detectable label, very small quantities of the target may be detected in the sample. Kits may further contain additional materials suitable for obtaining and testing a sample for the presence or absence of target such as suitable buffers, containers, and any needed instructions.

Another embodiment of the invention is directed to complexes comprising a nucleic acid to which additional targeting, therapeutic or labelling agents are coupled. In this embodiment, it is not necessary that the nucleic acid be the only targeting agent. Such targeting agents include, for example, chemicals, antibodies, antibody fragments, such as variable region portions, cell-associated antigens, binding proteins, or portions of binding proteins, receptor ligands, receptors and other molecules with a specific affinity to a target. The aptamer or additional nucleic acid may be used as a template for PCR or other amplification method (C. C. Sabayanayam et SPIE 3606: 90-97, 1999, the teaching of which is incorporated herein by reference) or as has been done immuno-PCR (T. Sano et al., Science 258: 120-122, 1992, the teaching of which is incorporated herein by reference).

Another embodiment of the invention is directed to a method for delivering an agent to a site by attaching the agent to a nucleic acid to which is coupled to a targeting agent. The nucleic acid complex is targeted to the site of interest via the targeting agent and delivers the agent. Using such methods, therapeutic agents can be targeted to diseased cells such as metastases, infected cells and microorganisms. Imaging agents can be targeted to specific sites in, for example, a human for imaging tissue. In addition, both imaging and therapeutic application are possible even though the site of the target may not be known. For example, all that is necessary to detect a metastatic cell is for the route of administration to administer the composition containing the complex into the area in which metastatic cells could arise (e.g. bloodstream, lymph, spinal fluid). Sufficient sensitivity can be achieved using appropriately detectable chemical moieties and therapeutic agents. As targeting is specific, damage to surrounding cells and tissues, if a concern, would be minimized.

The following examples are offered to illustrate embodiments of the invention, and should not be viewed as limiting the scope of the invention.

### EXAMPLES

### Example 1

### Validation of the Aptamer Approach

Experiments were undertaken to establish the usefulness of the aptamer approach in tumor targeting. CEA is a first target. CEA is, arguably, the best-studied tumor epitope and present on the largest number of tumors (For review, see Horig et al., 2000; El-Sadek et al., 2003, the teaching of which is incorporated herein by reference). This tumor-associated antigen is available commercially and there are many antibodies directed to this antigen. Further, a nude mouse CEA-expressing tumor model is available. The LS174T cell line, which grows well in nude mice, was used as a target for anti-CEA antibodies (D. J. Hnatowich et al., Nucl. Biol. Med. 36:7-13, 1992, the teaching of which is incorporated herein by reference). Cells are prepared for inoculation by growth in tissue culture. Cells grown in tissue cultures are used as an in vitro source of CEA pre-animal studies. Antibodies directed against CEA were among the first to be studied in patients. Anti-CEA antibodies were shown to be useful for the detection and therapy of colorectal cancers (D. M. Goldenberg et al., Sem. Nucl. Med. 19:262-81, 1990, the teaching of which is incorporated herein by reference). Patient trials have been conducted with several anti-CEA antibodies (D. J. Hnatowich et al., Cancer Res 50:7272-78, 1990, the teaching of which is incorporated herein by reference). The C110 anti-CEA IgG antibody labelled with 111In produced extremely good tumor images. Hence, the C110 antibody is a useful standard against which the aptamers may be compared.

CEA is a highly glycosylated cell-surface protein with a molecular mass of about 180 kD. The antigen is not expressed in normal adult cells but in embryonic cells although expression is increased on ˜50% breast, ovarian, colon and other cancer cells (e.g. Battifora and Kopinski, 1985, the teaching of which is incorporated herein by reference) (J. E. Shively et al, CRC Crit. Rev. Oncol. Hematol. 2:366-99, 1985, the teaching of which is incorporated herein by reference). Hence, CEA appears to be a general tumor marker.

These, and other conditions, lead to an increase in blood CEA; hence, clinically, serum CEA levels may be indicative (but not diagnostic) of the return of active metastatic disease. CEA belongs to the CEA superfamily, a subset of the immunoglobulin (10) superfamily with analogous constant and variable regions, (For reviews see Beauchemin and Kisilevsky, 1998; Gold et al., 1997, the teaching of which is incorporated herein by reference). The CEA gene family encodes 18 cross reacting proteins divided into a CEACAM branch (7 members) and the PSG branch (11 members) that are located, along with 11 pseudogenes, in two clusters on human chromosome 19 (19q13.1 and 19q13). In addition, a three-dimensional structure of CEA has been proposed (P. A. Bates et al., FEBS 301:207-14, 1992, the teaching of which is incorporated herein by reference). CEA belongs to a large family of proteins that include non-specific cross-reacting antigen (NCA), as well as other similar proteins. Thus, it is useful to eliminate aptamers which bind NCA or other molecules, both cell bound, and cell free, that are present in serum, NCA is difficult to obtain in pure form, but fortunately, it is expressed strongly on normal granulocytes (von Kleist et al., Proc. Natl. Acad. Sci. U.S.A. 59:2492-94, 1974, the teaching of which is incorporated herein by reference). Therefore, screening of the selected libraries with formed elements should remove aptamers with affinity for this antigen as has been done in the past by other developing CPA-binding monoclonal antibodies (HJ. Hansen et al., Cancer 71:3478-85, 1993, the teaching of which is incorporated herein by reference).

The spatial structure and localization of antigenic determinants on CEA has been determined (A. F. Pavlenki et al., Tumor Biol. 11:3006-118, 1990 Anti-CEA antibodies divide into 5 epitope groups, referred to as Gold 1-5 (Hammarstrom et al., 1989, the teaching of which is incorporated herein by reference). Note the conclusions of many studies are compromised because unknowingly anti-CEA antibodies cross-reacting with other CEA family proteins were used.

The application of this method to a large protein such as CEA can select a series of aptamers against different regions of the protein molecule. The use of multiple rounds of selection should ensure that the aptamers with highest affinities will be present, eventually, in the highest concentration.

### Example 2

### Initial Aptamer Libraries

Chemical composition: The aptamer approach has been successfully applied to DNA as well as RNA libraries. Thus, it appears that earlier concerns that DNA did not possess the structural diversity of RNA has been eliminated. Several technical reasons argue for the use of DNA aptamers libraries. For instance, the use of RNA in direct aptamer selection requires that the RNA can be converted to DNA before PCR amplification, and then the RNA is remade by transcription. The use of DNA eliminates the need for these steps. DNA is also much more chemically stable than RNA and easy to synthesize in bulk quantities.

The aptamer library used in the initial experiments was a pool of 100-mer ssDNAs (synthesized by Operon Technologies, Alameda, Calif.) with an internal 64 base variable region flanked by two 18 base constant sequences used as PCR primer sites (Left-1 5′-ATACCAGCTTCTTCAATT-3′ [SEQ ID NO. 1], and b-Right-1, 5′-biotin AGATTGCACTTACTATCT-3′ [SEQ ID NO. 2]) chosen because they lack secondary structure and do not anneal to each other (Crameria and Stemmer, 1993, the teaching of which is incorporated herein by reference). The 64 base variable regions allows theoretically ˜1038 potential sequences to be queried, however, biochemical considerations limit the SELEX library to ˜1014 distinct DNA sequences, so only a small fraction of potential aptamer sequences are queried in each SELEX experiment.

The chemically synthesized library was amplified in a 20 ml reaction with 300 pmole of library DNA, 0.6 microM each of both primers, 1× GeneAmp PCR buffer II (50 mM KCl, 10 mM Tris-Cl (pH 8.3), 1.9 mM MgCl2, 200 microM each dNTP's, 5 units/ml PerfectMatch PCR enhancer and 25 units/ml. Amplitaq Polymerase (Perkin-Elmer, Indianapolis, Ind.). The samples were incubated at 96 C for 8 min, followed by 20 cycles of 4 min at 94 C, 5 min at 46 C, 5 min at 72 C, and final extension for 20 min at 72 C. The products were phenol/chloroform extracted, precipitated with ethanol and dissolved in 2.0 ml of TE (10 mM Tris-Cl (pH 8.0), 0.1 mM EDTA). This PCR was done to obtain material for the selection protocol. A 50 microL portion of the library was re-amplified by PCR using the conditions described above, except the initial denaturation was at 95 C for 5 min, followed by 25 cycles of 1 min at 94 C, 1 min at 46 C, 1 min at 72 C and a final extension for 10 min at 72 C in a reaction volume of 3.7 ml. This PCR increased the number of each distinct sequence to ˜10 copies each. The 100 base pair (bp) double-stranded (ds) DNAs, purified from the MetaPhor (FMC Corp., Philadelphia, Pa.) agarose using a QIAEX II kit (QIAGEN, Valencia, Calif.), were used as template in a single sided PCR using only the Left-1 primer. The ssDNA products were purified through a streptavidin column (to remove the biotinylated right primer) then purified electrophoretically as described above. For detection purposes, purified ssDNAs were 5′-labeled with 32P using T4 polynucleotide kinase (New England Biolabs, Beverly, Mass.). The labelled ssDNAs were extracted with phenol/chloroform, precipitated with ethanol and dissolved in a binding solution representative of physiological conditions (150 mM NaCl, 3 mM MgCl2, 1 mM MnCl2, 0.1 mM CaCl2, 20 mM N-[2-Hydroxyethyl piperazine-N′-2-ethanesulfonic acid] (HEPES), pH 7.0).

### Example 3

### Affinity Purification

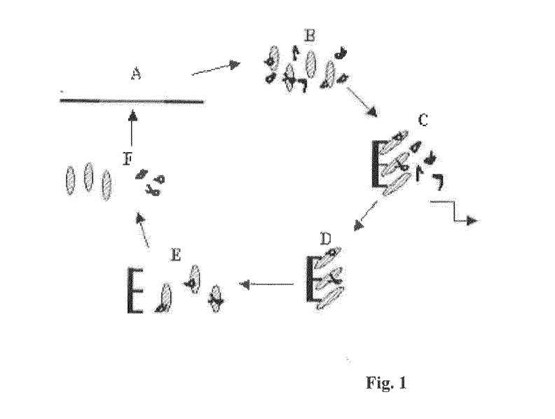

CEA (obtained from Calbiochem) was isolated from the supernatant of culture SW1116 human colon adenocarcinoma cells by affinity chromatography using anti-CEA antibodies. Affinity purification utilizes an immobilized CEA target to capture the highest affinity aptamers. Immobilization can be performed in several ways. The protocol used commercially available concanavalin A (ConA) column. Here, sugar residues present on CEA bind to ConA (S. Daniel et al., Int. J. Cancer 55: 303-10, 1993; David and Reisfeld, 1974, the teachings of which are incorporated herein by reference). The CEA-DNA complex can be eluted from the ConA columns with an excess of alpha-methylmannoside and/or alpha-galactoside. The final double selection protocol that yielded anti-CEA aptamers is shown in FIG. 1.

FIG. 1. Double SELEX. Protocol for Isolation of anti-CEA aptamers. (A) DNA library was amplified and labelled with 32P (B) Labelled DNAs were heated to 90 C for 5 min, slowly cooled to ˜22 C and mixed with 200 micrograms of CEA in binding solution at room temperature with gentle mixing for 30 min. (C) The DNA/CEA mixture was applied to a 500 microL Con-A column (Amersham-Pharmacea, Indianapolis, Ind.) equilibrated with binding buffer (D) Unbound ssDNA was removed by washing the column with binding solution. (E) CEA/DNA complexes were eluted from the ConA with 0.5 M alpha-methyl-D-mannoside and 0.5 M alpha-methyl-D-glucoside dissolved in binding buffer. These sugars disrupt the interaction between the CEA and the immobilized Con-A. (F) Aptamers were purified from CEA by phenol extraction, precipitated, amplified by PCR, labelled, then subjected to another round of SELEX. The amounts of 32P-DNA input, and bound to, and then eluted from the columns were determined by Cerenkov counting. Negative selection cycles, done in the absence of CEA, were used to eliminate DNAs bound to non-CEA components of the column. The unbound fractions were collected for the negative selection cycles.

### Example 4

### The Enriched Aptamer Library

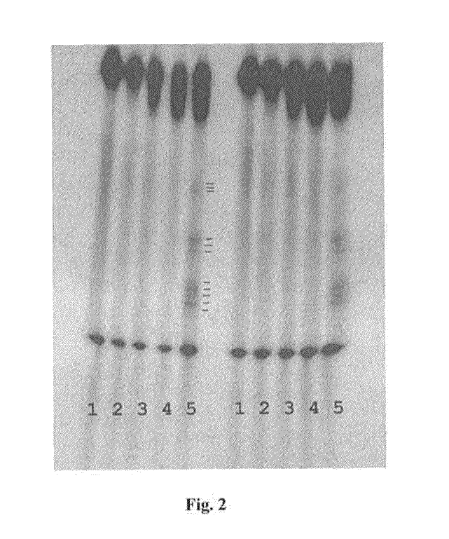

In total, eight CEA binding cycles and two negative selection cycles were performed (Table 2). Ultimately, 21% of the library bound to CEA, which would represent a Kd of ˜10-6 M if this were a single aptamer molecule. An indirect end-labelling experiment was done to confirm the selection of a reduced complexity library after round 5 (FIG. 2). In these experiments, the aptamer library, labelled at the 5′ end, was digested with 3 restriction enzymes with 4-base recognition sites. Initially, cleavage of the input DNA of round 1 with high complexity (i.e. ˜1014 different molecules) produced a smear, but when the complexity (i.e. the number of different fragments in the pools) was reduced (round 5) specific hands were observe.

TABLE 2
Percent oligonucleotide library binding to CEA after sequential
cycles of SELEX. The % bound was determined by measuring the
amount of 32P-labeled DNA bound to CEA immobilized on the ConA
column. In the first cycle, a high level of non-specific binding
was observed; hence, the flow through, rather than the bound
DNA, was used as input into cycle 2 even though the bound fraction
likely contained specific anti-CEA aptamers. The flow-through
was also used from cycles 7 and 8 because the retained molecules
bound to the column in the absence of CEA.

| | CEA | DNA | Bound |
|---|---|---|---|
| Cycle | (microgram) | (microgram) | (Percent) |
| 1 | 200 | 76 | 5.99 |
| 2 | 200 | 27 | 0.91 |
| 3 | 200 | 8 | 0.42 |
| 4 | 200 | 10 | 0.61 |
| 5 | 200 | 9 | 2.93 |
| 6 | 200 | 6.7 | 18.9 |
| 7 | 0 | 3.3 | — |
| 8 | 0 | 3.3 | — |
| 9 | 200 | 2.9 | 9.9 |
| 10 | 200 | 3.0. | 21.1 |

FIG. 2. Analysis of library complexity by following the appearance of specific restriction fragments from end-labelled DNA pools. Duplicate fractions of DNA pools after the indicated selection cycle (number in figure) were PCR amplified using a 5′-32P end-labelled left primer. The gel purified 100-mer PCR products, digested with restriction enzymes (Sau3A I (′GATC), Hinf I (G′ANTC) and Aci I (C′CGG)) not cleaving within the constant end sequences, were fractionated by denaturing PAGE (8% polyacrylamide gel, 7 M urea) that was exposed 10% Kodak BioMax MR film. In these experiments, only one fragment from each DNA is visible, i.e. the fragment containing the label, and all input DNA is the same size (100 mer). Binomial statistical calculations predict 96% of random 64-mers will have at least one cleavage site. The data demonstrated that most oligonucleotides were not cleaved by any of the restriction enzymes because the majority of the end-label is at the top of the gel where the 100-mer fragment runs. At the bottom of the each lane is unincorporated labelled primer. The appearance of specific bands (indicated with a horizontal line), between the primer and the 100-mer DNA, after round 5, was indicative of a low complexity DNA pool. In the earlier cycles, shorter fragments may have been present but none were at a high enough concentration to be visible.

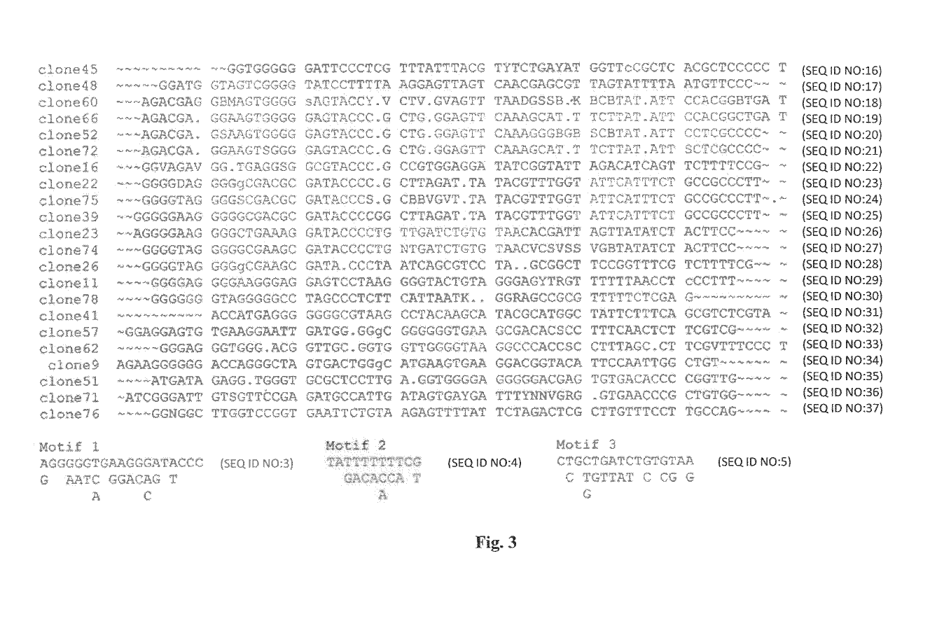

The oligonucleotides bound to CEA after cycle 10 were cloned into plasmid pCR-Blunt (Invitrogen, Carlsbad, Calif.) using Escherichia coli strain top10. Fifty-eight randomly chosen clones were sequenced using standard methods. An initial sequence comparison performed with the Pile-Up (Wisconsin Group GCG package) revealed many sequences that were isolated multiple times and 22 unique sequences (FIG. 3). A comparison of the unique sequences with MAST and MEME (San Diego Supercomputing Center) surprisingly identified three consensus motifs. Motif 1 and 3 weakly share a core sequence rich in G nucleotides, motif 13. A high number of G-residues is characteristic of native and modified ssRNA and ssDNA aptamers that form intra- or inter-molecular structures (Shafer and Smirnov, 2001) G-quartets (Gold et al., 1995, the teaching of which is incorporated herein by reference).

FIG. 3. Unique first-generation anti-CEA aptamers and consensus sequences. Aptamer sequences were aligned using the Wisconsin Groups GCG package, pile-up algorithm. Of the original 59 clones sequenced, 22 were unique. Further analysis using Meme/Mast uncovered 3 conserved motifs, motif1-AGGGGGTGAAGGGATACCC [SEQ ID NO: 3](green), motif 2 TATTTTTTTCG [SEQ ID NO: 4](red), and motif 3-CTGCTGATCTGTGTAA [SEQ ID NO: 5](blue). Motif 1 and 3 share a core motif (GGTGAA) [SEQ ID NO: 6] called motif 13. Three clones had no motifs. Ambiguous bases obtained in the sequencing experiments are indicated using the IUPAC-IUBMB nomenclature: (R (U or A), Y (T or C), M (A or C), K (U or T), S (U or C), W (A or T), H (A or C or T), B (G or T or C), V (O or C or A), D (U or A or T), N (U or A or T or C)).

The results of the sequence analysis revealed several unusual occurrences. First, the motif-1 occurrence usually occurred nearby the 5′ primer. Note that the analysis revealed several unexpected results. For instance, usually only one or two conserved motifs are found, usually there is one motif/oligonucleotide, and the motif is located in a variable position in the variable region.

### Example 4

### Characterization of Isolated Aptamers In Vitro

A fluorescent polarization (FP) assay was used to test the affinity of several oligonucleotide sequences to CEA (Table 4). The initial studies focused on 5 oligonucleotides. Clone 2.2 and clone 26 contained the sequences with the highest identity to the G-rich motif. The aptamers were synthesized with fluorescein at their 5 ends and binding was analysis using the Beacon 2000 instrument equipped with a 530 nm emission filter and a 490 nm excitation filter from Pan Vera Corp. (Madison Wis.).

TABLE 4
G-rich consensus and oligonucleotides sequences tested in a fluorescent
polarization (FP) assay. The G-rich consensus was developed from 21
occurrences in the library shown in Table 3 including data from the three
clones (clones 66, 52 and 72) having two occurrences. Note that R = A or G.
Bases highlighted in blue deviate from the consensus.

| Consensus | Level of conservation (%) | |
|---|---|---|
| GGGNNGGGGNNGNNGNNNTACCC | 60 | [SEQ ID NO: 7] |
| GGGGAGGGGGNGNNGGGATACCC | 50 | [SEQ ID NO: 8] |
| GGGGGAGGGGGTGRGGGATACCCC | 40 | [SEQ ID NO: 9] |

Oligonucleotides studied
ATACCAGCTTATTCAATTGGGGTAGGGGGCGAAGCGATACCCTAATCAGC
clone26 bases 1-50 [SEQ ID NO: 10]
GGGGTAGGGGGCGAAGCGATACCCTAATCAGC
[SEQ ID NO: 11]
ATACCAGCTTATTCAATTGGGGGAGGGGGCGACGCGATACCC
clone22 bases 1-42 [SEQ ID NO: 12]
GGGGGAGGGGGCGACGCGATACCC
clone22 bases 19-42 [SEQ ID NO: 13]
ATACCAGCTTATTCAATT
5′ primer [SEQ ID NO: 14]
CGGGAATTCTGGCTCTGCGACATGA
random sequence [SEQ ID NO: 15]

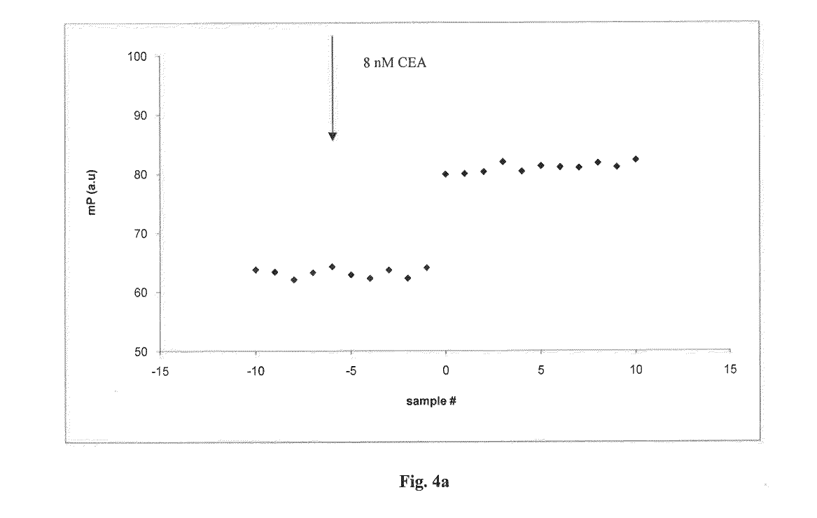

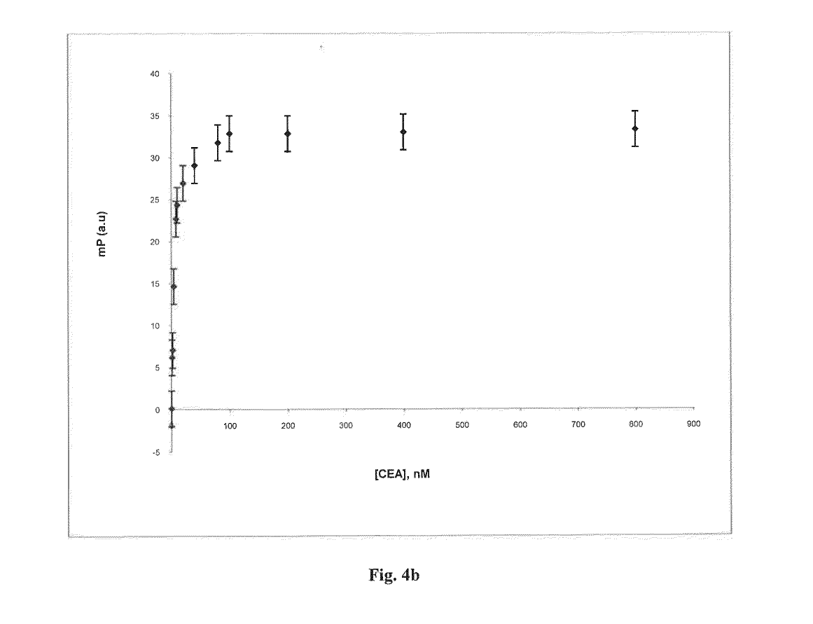

In the FP assay, fluorescently labelled aptamers are added to the protein. Binding is detected by an increase in fluorescent polarization cause by the decrease in rotation of the protein bound fluorescein (FIG. 4A). The Kd is calculated by determining the CEA concentration at the half maximal mP value as determined from plots of averaged stable mP data as shown in FIGS. 4B and C. Kd's are summarized in Table 4. The aptamers and the primer itself show good binding with CEA having a Kd at 4 nM for clone26bases1-50 and 5 nM for clone22bases1-42 and the primer. The absence of the primer increased the Kd to 20 nM. Unfortunately, others members of the CEA superfamily are not available commercially. Hence control proteins used were bovine serum albumin (BSA) and bovine gamma globulin (BGG) and Kd was found in the 100-500 nM concentration range. The random primer showed no binding to CEA in the 2-800 nM.

In summary, the results thus far show that two distinct sequences were identified that have high affinity and specificity for CEA. The sequence with the highest affinity and specificity was the original 5 primer. The conserved G-rich motif also has high affinity and specificity for CEA.

FIG. 4. Binding of clone26bases1-50 to CEA monitored by FP in a physiological binding buffer using 4 nM aptamer. (A) Shown is the mP as a function a reading number (sample #). CEA (8 nM) was added after the tenth reading. (B) Shown is the average mP vs aptamer concentration. (C) Shown is the average mP vs CEA concentration within the linear range.

TABLE 3
The dissociation constants (Kd) for the oligonucleotide binding
to the CEA and other control proteins BSA and BGG.

| | K_{d} (nM⁻¹) | | |
|---|---|---|---|
| Oligonucleotide | CEA | BSA | BGG |
| [Fl]clone26bases1-50 | 4 | 100 | 100 |
| [Fl]clone22bases1-42 | 5 | 200 | 500 |
| [Fl]clone22bases19-42 | 20 | 400 | 250 |
| [Fl]5′-Primer | 5 | 300 | 400 |
| [Fl]random Primer | >800 | not done | not done |

### Example 5

### Characterization of Anti-CEA Aptamers Using Whole Cells In Vitro

The ability of fluorescently labelled aptamers to bind to tumor cell-bound CEA under conditions approximating that in vivo will be tested using cultured cells. The LS174T tumor line can be grown in culture routinely. Cells will be harvested and suspended in binding buffer or fresh serum. Suspended sells will be incubated at 37 C with the labelled aptamer under investigation and the kinetics of binding established by sampling at multiple time points. Cells in each sample can be separated by centrifugation, and counted after washing or by FACS analysis.

Oligonucleotides with affinity for NCA is identified by testing which fresh human whole blood since NCA is heavily expressed on normal granulocytes (von Kleist et al., Proc. Natl. Acad. Sci. U.S.A. 69:2492-94. 1974, the teaching of which is incorporated herein by reference), This assay may be complicated if the aptamers bind to serum proteins. If necessary, negative rounds of secondary rounds of selection will be done with purified granulocytes and/or serum.

The solution-based binding protocol allows the experiments to be conducted easily at 37 C and at physiological salt concentrations to imitate in vivo conditions as closely as possible. Environmental conditions are also certain to modulate molecular conformation and/or affinity binding. In fact, CD analysis showed that CEA undergoes a reversible conformation transition between 20-55 C. At temperatures above 55 C, an irreversible conformation change occurs.

The highly negative charge of DNA and the conformation of ssDNA is almost certainly influenced by the nature and concentration of cations such as magnesium ions, these binding studies can also be performed in tissue culture media such as Gibco 1640 or the equivalent mixed with calf serum. This mixture can be used with divalent metal ions at concentrations similar to that found in human serum.

### Example 7

### Optimizing Aptamer Sequences

After selecting the best aptamer sequence, further optimization will be done, if necessary, by an approach conceptually similar to the in vivo process of somatic hypermutation that improves immunoglobulin affinities. The aptamer optimization protocol involves randomly mutagenizing the best aptamer sequence(s) in a mutagenic PCR reaction and reselecting the high affinity aptamer to create a second-generation library (B. Borrego, A. Wienecke, A Schweinhorst, Nucleic Acids Res. 23: 1834-1845, 1995; P. S. Chowdhury and I. Pastan, Nature Biotech. 17: 568-572, 1999; W. P. C. Stemmer. Proc. Nat. Acad. Sci 91P 10747-10751, 1994; W. P. C. Stemmer. Nature 370, 1994, the teachings of which are incorporated herein by reference). Random mutagenesis is done using an error prone PCR where Mn+ is substituted for Mg+ to promote insertion of mismatched bases (1). W. Leung and D. V. Goeddel. J. Methods Cell and Mole Bio. 1: 11-15, 1989, the teaching of which is incorporated herein by reference). Then the mutagenized and amplified sequences are subjected to several rounds of SELEX as described above. The SELEX protocol that will be used is the same as used above except that ‘affinity competition’ will be used to isolate aptamers with affinities higher than the first-generation aptamers and cell-bound CEA will be used as a target. In the affinity competition approach, CEA or tumor cells are incubated with mutagenized and labelled sequences and then immobilized on a ConA column as before. The column is washed with the first-generation library to remove DNAs with affinities that are <the first-generation aptamers before the CEA is released from the column by 0.5 M alpha-methyl-D-mannoside and 0.5 M alpha-methyl-D-glucoside. A model system using free biotin to compete off biotinylated ssDNA bound to streptavidin coated beads demonstrated the feasibility of this approach and was based on earlier experiments demonstrating free biotin bound streptavidin with higher affinity than biotinylated macromolecules (unpublished observations). This system also demonstrated that his-biotinylated ssDNA have greater affinities than monobiotinylated ssDNA for streptavidin.

The use of whole cell targets means that the bound synthetic DNAs will be part of a large pool of intracellular nucleic acids (90% RNA, 10% DNA) after cell lysis and phenol extraction. It may be necessary to treat the cell extracts with DNase free RNase A to remove RNA, or to pre-purify the oligonucleotides by capture on streptavidin coated magnetic beads. A streptavidin capture protocol would require a single amplification step using a biotinylated right primer. The amount of DNA binding will be determined and the complexity of the pools after each round of selection will be followed as done earlier. When, no further increase in binding is detected, the pool of aptamers will be cloned, sequenced, the consensus re-established and tested as described.

### Example 8

### In Vivo Anti-CEA Aptamer Experiments

The behavior of the aptamers can be characterized in vivo. These studies focus on determining the biodistribution and stability in normal mice. Ultimately, the ability of a subset of the radio-labelled aptamers to target CEA-expressing tumors in nude mice can be measured and compared relative to that of a radiolabeled anti-CEA antibody.

The pharmacokinetics of each 32P or 111In-labeled aptamer can be determined in normal CD-1 male mice (Charles River, Wilmington, Mass.) by measuring the biodistribution at least three time points. Each animal receives about 151 micrograms (i.e. about 0.6 mg/kg body weight) of aptamer (about 15 microCi of a radionuclide label) via a tail vein. Animals are sacrificed at 1, 4 and 24 hour time points and a biodistribution determined at each time point (G. Mardirossian et al., Nucl. Med. Comm. 13:503-12, 1992; D. J. Hnatowich et al., Nucl. Med. Comm. 14:52-63, 1993; J. Nucl. Med: 109-19, 1993; Nucl. Med. Biol. 20:189-95, 1993, the teachings of which are incorporated herein by reference). Aptamers can also be radiolaheled with 99mTc via the ShNH moiety (D. J. Hnatowich et al., J. Nucl. Med. 36:2306-14, 1995, the teaching of which is incorporated herein by reference) and the biodistribution at the same time points compared with that of the 111In-labeled aptamer. In both cases, serum and urine samples and homogenates of liver and kidney tissues can be analyzed by size exclusion HPLC (DJ. Hnatowich et al., Nucl. Med. Comm. 14:52-63, 1993, the teaching of which is incorporated herein by reference) or paired-ion HPLC (H. Sands et al., Mol. Pharmacol. 45:932-43, 1994, the teaching of which is incorporated herein by reference) as has been used to determine the chemical form of each label in these tissues and fluids. An important object of these measurements is to establish the stability of the aptamer and its label. It will be apparent by one or another of the above HPLC techniques whether the aptamer remains intact in these tissues, and whether the label remains attached.

Tumor binding in vivo: Those aptamers or modified aptamers displaying high affinities for CEA, low binding to formed elements and serum proteins, stability to nucleases in serum and low levels of accumulation in normal organs can be investigated in tumor-bearing nude mice. The LS1741T tumor system has been used by many investigators for tumor targeting in animal studies. This tumor expresses CEA (D. H. Hnatowich et al., Nucl. Med. Comm. 14:52-63, 1993; Nucl. Med. Biol. 20:189-95, 1993, the teaching of which is incorporated herein by reference). The LS174T cells are maintained in culture and prepared for administration. Approximately 106 cells are implanted subcutaneously in a flank. Approximately 10-14 days later, the tumors have reached a size of about 1 cm in their largest diameter. At this time, each animal receives about 15 micrograms of one labelled. The positive control in these experiments, anti-CEA antibody will be radiolabeled the Differential labelling of the aptamer and the antibody will allow the behavior of both molecules to be followed simultaneously. The principal object of these studies is to evaluate the properties of the labelled aptamer for tumor targeting relative to that of a successful anti-tumor antibody. Bio-distributions can be determined at 1, 4 and 24 hour intervals as before.

Imaging studies can also be performed with 99m-Tc labelled aptamer and 111In labelled antibody. using Elscint 409M camera. In these cases, the animals will have received a single radionucleotide because of the interference of 111In or photons in the 99mTc window.

### Example 9

### Validation of PNA Applications

A novel group of oligonucleotides have been described which are referred to as PNA of peptide nucleic acids wherein the phosphodiester bonds of the nucleic acid backbone were replaced with amide (peptide-like) bonds (P. E. Nielsen et al., Sci. 254:1497-1500, 1991, the teaching of which is incorporated herein by reference). These derivatives show extremely interesting characteristics. For example, although capable of hybridizing to their complement in a fashion similar to natural oligonucleotides, PNAs are neutral, optically inactive, and extremely stable in vivo (S. C. Brown et al., Sci. 265:777-80, 1994; O. Bouchard et al., Trends in Biotech. 11:384-86, 1993, the teachings of which are incorporated herein by reference). As PNAs can be radiolabeled by known methods, use of PNAs in the aptamer approach described above is realistic and practical. However, strategies for the development of single-stranded PNA (ssPNA) aptamers are considerably more difficult than strategies using ssDNA aptamers. For example, although PNAs are synthesized using standard protein chemistry, the cost is very high. Although a PNA library could be selected in the same manner as DNA or RNA library, PNA cannot be amplified by PCR. Hence, the sequences of selected ssPNAs can only be determined indirectly, for example, by identifying DNA molecules that are complementary to PNAs. The DNAs can then be sequenced using standard methods and other reduced complexity PNA libraries designed based on the PNA consensus sequences.

### Example 9

### Multivalent Aptamers

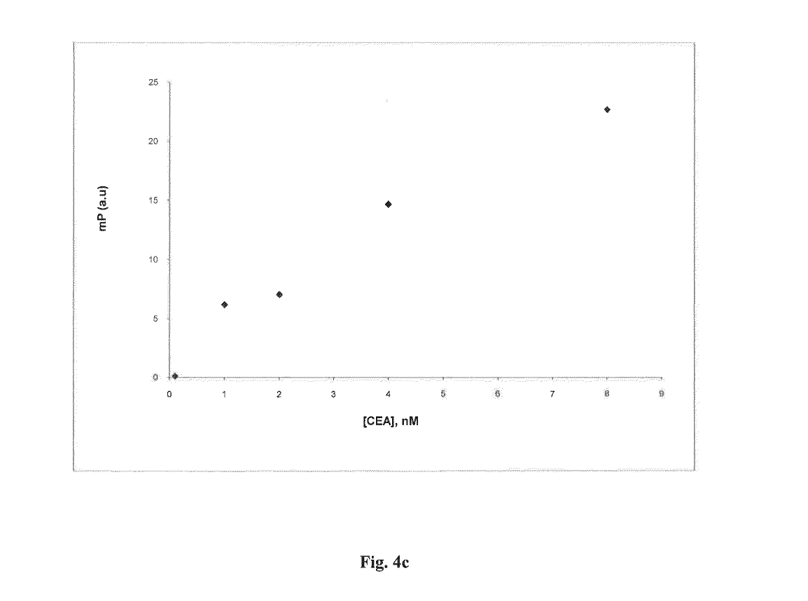

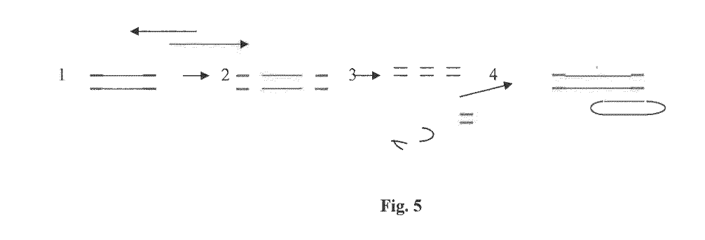

Nature uses multivalency to enhance affinities and specificity between molecules. A well-known example is IgM where 10 low affinity single antigen-binding sites are combined into a single complex to produce a high affinity antibody. Although enhanced binding of bivalent antibody fragments against CEA was demonstrated (Robert et al., 1999; Wu et al., 1996; Wu et al., 1998, the teachings of which are incorporated herein by reference), construction of functional multivalent molecules is not simple. Many times linkage of two binding sites hinders rather than enhances binding and many efforts have failed. A heterogeneous bivalent anti-CEA antibody (Robert et al., 1999, the teaching of which is incorporated herein by reference) made up of Fab′ fragments binding to different CEA epitopes has an affinity for CEA ˜10-fold greater than the monomers. Bivalent aptamers against human neutrophil elastase (H N E Davis et al., 1996, the teaching of which is incorporated herein by reference), and thermostable DNA polymerase (Lin and Jayasena, 1997, the teaching of which is incorporated herein by reference) have been chemical synthesized. The bivalent anti-HNE molecule, composed of DNA binding sequences and a weak competitive inhibitor tetrapeptide, was 105 more effective than the tetrapeptide alone. The anti-polymerase aptamers effectively inhibited the different enzymes bound by the monomer units. Generally bivalency in designed molecules has increased affinity 10-fold. However, recent experiments selecting bifunctional molecules have demonstrated even greater enhancements. For instance, Burke and Willis (1998) combined two low affinity aptamer sequences using overlapping primer regions that are extended in a mutagenic PCR to produce bivalent aptamers against Coenzyme A, chloramphenicol and adenosine (FIG. 5A). Bittker et al. (2002) selected anti-streptavidin aptamers through non-homologous in vitro recombination (FIG. 5B). The former method is simple but the later method selects the appropriate linker sequences as well as heterogeneous aptamers for increasing specificity or functionality in vivo.

FIG. 5. Schematic of multivalent selection methods described by (A) Burke and Willis (1998) and (B) Bittker et al. (2002). In the former method, (1) the DNA library was treated with a restriction enzyme to remove the biotinylated primer ends, (2) DNase to introduced random nicks and T4 DNA polymerase to produce blunt ended fragments, (3) DNA ligase to create chimeric double stranded molecules (hairpin oligonucleotides incorporated into the growing dsDNA controlled the length of the recombinant molecules). (4) The hairpin DNA was removed by digestion with a restriction enzyme before the selection step and the primer sequence added by ligation after the selection step.

Thus far, all multivalent aptamers have been single stranded molecules with end aptamer sequences. The single stranded backbone provides maximum flexibility to the molecules. Note that greater rigidity may lead to greater affinity. For instance, the extraordinary high affinity of streptavidin for biotin is at least partially due to the great rigidity of biotin, (e.g. as is seen the biotin streptavidin interactions). Rigidity can be built into aptamers using double stranded regions or even circular molecules (see FIG. 5).

The structural properties of DNA include the persistence length (of ssDNA vs dsDNA being ˜4 bases vs ˜400 bp, respectively), the helicity of duplexed DNA (10.4 base pairs per turn), the rise of a single base (3.4 nm). PolyA/T stretches or gaps (Guo and Tullius, 2003, the teaching of which is incorporated herein by reference) induce bends in double stranded DNA. Mismatches placed near the hinge would provide some flexibility in the duplex (Cantor et al., 1999, the teaching of which is incorporated herein by reference). The common schematic of an antibody structure is shown in FIG. 5. Nucleic acid molecules with similar, less structures can be constructed from nucleic acids that are partially double stranded duplexed regions provide rigidity and spatial limitations and could prevent interaction of the linker with the aptamer sequence.

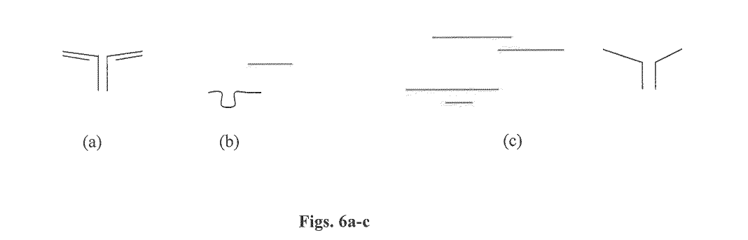

The best compromise between rigidity and flexibility may be the partially duplexed molecules depicted in FIG. 6C. These molecules do not require the synthesis of long nucleic acids to make multimeric species, and an individual biotinylated single strand could be isolated on streptavidin coated heads, mutagenized, denatured and reannealed to the second strand to recreate the chimeric molecules. Here and elsewhere the rapid prototyping capabilities of DNA molecules are quite useful.

FIG. 6. Schematic of a bivalent (A) antibody and potential (B) single stranded (C) double stranded aptamers. Here, the binding moieties are located at the ends of the indicated DNAs. Note that duplexed DNA regions are rigid and control orientation of single stranded tails.

Multivalent anti-CEA aptamers may already be in hand because the experiments described above revealed at least two sequences on the same molecules that independently bind CEA. Competition experiments will reveal whether the two aptamer sequences bind to the same target and whether both binding sites are active when present on the same molecule. It is likely that the aptamers with multiple binding sites will need to be optimized by reselection. Multivalent aptamers may be created by combining the aptamers identified by us or others (e.g., the short DNA GRO sequence or other DNA aptamers or other sequences like the long anti-xLEX RNA). If two different cell epitopes are targeted then, by necessity, the selection/testing protocol would need to be done on tumor cells that express both epitopes or an artificial system like magnetic beads that has both epitopes present on their surface. The initial experimental goal is to determine whether the affinity of the bivalent aptamer for tumor cells or beads is greater than affinity of monovalent aptamers. In the case of the GRO, the effectiveness of inhibition of tumor cell growth will be monitored as has been done for GROs (Bates et al., 1999, the teaching of which is incorporated herein by reference). For in vitro testing of cells, each monomeric aptamer sequence would be testing as a competitor of the multimeric aptamer and also serve to select effective multimeric species. Here, for instance biotinylated monomeric aptamer can be removed from a pool of DNAs using streptavidin coated beads, leaving only multimeric species. Alternatively, PCR with primers only present on multimeric species could be used to amplify the multivalent target molecules. In these experiments, the linker sequence and an aptamer sequence optimized for bivalent molecules.

Another method to create multivalent aptamers would involve using an identified anti-CEA aptamer sequence linked to a random DNA sequence. Here, the second binding sequence as well as the linker will be selected de novo.

All documents disclosed herein, including the sequence listing attached in both paper and computer-readable format, are specifically incorporated by reference.

What is claimed is:

1. An aptamer that binds to CEA, wherein the aptamer comprises a consensus nucleic, acid sequence selected from the group consisting of SEQ ID NOs: 3, 4, 5, 6, 7, 8, and 9.

2. The aptamer of claim 1, wherein the aptamer comprises a nucleic acid sequence selected from the group consisting of SEQ ID NOs: 10, 11, 12, 13, 16, 17, 18, 19, 20, 21, 22, 23, 24, 25, 26, 27, 28, 29, 30, 31, 32, 33, 34, 35, 36, and 37.

3. The aptamer of claim 1, having a dissociation constant of about 4 nM to about 20 nM.

4. The aptamer of claim 1, wherein the aptamer comprises single-stranded DNA, RNA or PNA.

5. The aptamer of claim 1, wherein the aptamer comprises natural or non-naturally occurring bases.

6. The aptamer of claim 1, wherein the aptamer comprises a chemical backbone selected from the group consisting of a phosphodiester, a phosphorothioate, a methylene phosphorothioate, a peptide and other chemical modifications.

7. The aptamer of claim 5, wherein the non-naturally occurring bases are selected from the group consisting of methylinosine, dihydrouridine, methyl guanosine and thiouridine.

8. A method of identifying, detecting, or imaging cells or tissues expressing CEA, the method comprising administering to a subject the aptamer of claim 1.

9. The method of claim 8, wherein the aptamer comprises a nucleic acid sequence selected from the group consisting of SEQ ID NOs: 10, 11, 12, 13, 16, 17, 18, 19, 20, 21, 22, 23, 24, 25, 26, 27, 28, 29, 30, 31, 32, 33, 34, 35, 36, and 37.

10. The method of claim 8, wherein the aptamer is coupled to an imaging agent selected from the group consisting of an isotope, a metallic radionuclide, a radioisotope, a magnetic substance, ara electro-magnetic substance and enzymes.

11. A pharmaceutical composition comprising a therapeutically effective amount of the aptamer of claim 1.

12. The composition of claim 11, wherein the aptamer comprises a nucleic acid sequence selected from the group consisting of SEQ ID NOs: 10, 11, 12, 13, 16, 17, 18, 19, 20, 21, 22, 23, 24, 25, 26, 27, 28, 29, 30, 31, 32, 33, 34, 35, 36, and 37.

13. A method of treating or detecting a disorder comprising administering to a subject in need thereof the composition of claim 11.

14. The method of claim 13, wherein said disorder is a neoplastic disorder selected from the group consisting of a small cell lung cancer, other lung cancer, rhabdomyosarcomas, choriocarcinomas, glioblastoma multiforme, brain tumor bowel carcinomas, gastric carcinomas, leukemias, ovarian cancer, breast cancer, prostate cancer, osterosarcomas, breast cell carcinomas, melanomas, hematologic melanomas, ovarian carcinomas, pancreatic cancers, liver cancers, stomach cancers, colon cancer, squamous cell carcinomas, neurofibromas, testicular cell carcinomas and adenocarcinomas.

15. The method of claim 13, wherein said disorder is selected from the group consisting of an immune system disorder, non-Hodgkin's lymphomas, follicular lymphomas, Burkitt's lymphoma, adult T-cell leukemias, adult T-cell lymphomas, hairy cell leukemia, acute myelogenous leukemia, lymphoplastic leukemias, chronic myelogenous leukemias and myelodysplastic, syndromes.

16. The method of claim 13, wherein the composition is administered in combination with another therapeutic agent.

17. The method of claim 16, wherein said therapeutic agent is selected from the group consisting of cells, nanoparticles, hormones, vaccines, haptens, toxins, enzymes, immune system modulators, anti-oxidants, vitamins, functional agents of the hematopoietic system, proteins, nucleic acids, metals, inorganic substances, virus particles, antigens, amino acids, peptides, saccharides, polysaccharides receptors, radioisotopes, radionuclides, stable isotopes, paramagnetic compounds, and pharmaceutical compounds.

18. A method of treating a neoplastic disorder comprising administering to a subject in need thereof a composition comprising an isolated oligonucleotide of sequence SEQ ID NO: 14 wherein said oligonucleotide binds to CEA with a dissociation constant of about 5 nM.

19. The method of claim 18, wherein said oligonucleotide comprises a chemical backbone selected from the group consisting of phosphodiester, a phosphorothioate, a methylene phosphorothioate, a peptide and other chemical modifications.

20. The method of claim 18, wherein said neoplastic disorder is selected from the group consisting of breast cancer, prostate cancer, cervical cancer, lung cancer and tumor cells that express CEA.


SEQUENCE LISTING (37 sequences)

SEQ ID NO: 1 - DNA, 18 bp, Homo sapiens
ataccagcttcttcaatt

SEQ ID NO: 2 - DNA, 18 bp, Homo sapiens
agattgcacttactatct

SEQ ID NO: 3 - DNA, 19 bp, Homo sapiens
agggggtgaagggataccc

SEQ ID NO: 4 - DNA, 11 bp, Homo sapiens
tatttttttcg

SEQ ID NO: 5 - DNA, 16 bp, Homo sapiens
ctgctgatctgtgtaa

SEQ ID NO: 6 - DNA, 6 bp, Homo sapiens
ggtgaa

SEQ ID NO: 7 - DNA, 23 bp, Homo sapiens
  misc_feature (4)..(18): wherein, n is a, c, g or t
gggnnggggnngnngnnntaccc

SEQ ID NO: 8 - DNA, 23 bp, Homo sapiens
  misc_feature (11)..(14): wherein, n is a, c, g or t
ggggagggggngnngggataccc

SEQ ID NO: 9 - DNA, 24 bp, Homo sapiens
  misc_feature (14)..(14): wherein, r is a or g
gggggagggggtgrgggatacccc

SEQ ID NO: 10 - DNA, 50 bp, Homo sapiens
ataccagcttattcaattggggtagggggcgaagcgataccctaatcagc

SEQ ID NO: 11 - DNA, 32 bp, Homo sapiens
ggggtagggggcgaagcgataccctaatcagc

SEQ ID NO: 12 - DNA, 42 bp, Homo sapiens
ataccagcttattcaattgggggagggggcgacgcgataccc

SEQ ID NO: 13 - DNA, 24 bp, Homo sapiens
gggggagggggcgacgcgataccc

SEQ ID NO: 14 - DNA, 18 bp, Homo sapiens
ataccagcttattcaatt

SEQ ID NO: 15 - DNA, 25 bp, Homo sapiens
cgggaattctggctctgcgacatga

SEQ ID NO: 16 - DNA, 59 bp, Homo sapiens
  misc_feature: wherein, y is t or c
ggtggggggattccctcgtttatttacgtytctgayatggttccgctcacgctccccct

SEQ ID NO: 17 - DNA, 63 bp, Homo sapiens
ggatggtagtcggggtatccttttaaggagttagtcaacgagcgttagtattttaatgttccc

SEQ ID NO: 18 - DNA, 64 bp, Homo sapiens
  misc_feature: wherein, b is g, t or c
agacgaggbmagtggggsagtaccyvctvgvagtttaadgssbkbcbtatattccacggbtgat

SEQ ID NO: 19 - DNA, 63 bp, Homo sapiens
agacgaggaagtgggggagtacccgctgggagttcaaagcatttcttatattccacggctgat

SEQ ID NO: 20 - DNA, 62 bp, Homo sapiens
  misc_feature: wherein, b is g or t or c
agacgagsaagtgggggagtacccgctgggagttcaaagggbgbscbtatattcctcgcccc

SEQ ID NO: 21 - DNA, 61 bp, Homo sapiens
  misc_feature: wherein, s is g or c
agacgaggaagtsggggagtacccgctgggagttcaaagcatttcttatattsctcgcccc

SEQ ID NO: 22 - DNA, 64 bp, Homo sapiens
  misc_feature: wherein, s is g or c
ggvagavggtgaggsggcgtacccgccgtggaggatatcggtattagacatcagttcttttccg

SEQ ID NO: 23 - DNA, 64 bp, Homo sapiens
  misc_feature: wherein, d is g or a or t
ggggdagggggcgacgcgataccccgcttagattatacgtttggtattcatttctgccgccctt

SEQ ID NO: 24 - DNA, 64 bp, Homo sapiens
  misc_feature: wherein, b is g or t or c
ggggtaggggscgacgcgatacccsgcbbvgvttatacgtttggtattcatttctgccgccctt

SEQ ID NO: 25 - DNA, 66 bp, Homo sapiens
gggggaagggggcgacgcgataccccggcttagattatacgtttggtattcatttctgccgccctt

SEQ ID NO: 26 - DNA, 64 bp, Homo sapiens
aggggaaggggctgaaaggatacccctgttgatctgtgtaacacgattagttatatctacttcc

SEQ ID NO: 27 - DNA, 62 bp, Homo sapiens
  misc_feature: wherein, n is g or a or t or c
gggtagggggcgaagcgatacccctgntgatctgtgtaacvcsvssvbgtatatctacttcc

SEQ ID NO: 28 - DNA, 62 bp, Homo sapiens
ggggtagggggcgaagcgataccctaatcagcgtcctagcggcttccggtttcgtcttttcg

SEQ ID NO: 29 - DNA, 62 bp, Homo sapiens
  misc_feature: wherein, y is t or c
ggggaggggaagggaggagtcctaaggggtactgtagggagytrgttttttaacctcccttt

SEQ ID NO: 30 - DNA, 55 bp, Homo sapiens
  misc_feature: wherein, k is g or t
gggggggtagggggcctagccctcttcattaatkggragccgcgtttttctcgag

SEQ ID NO: 31 - DNA, 60 bp, Homo sapiens
accatgagggggggcgtaagcctacaagcatacgcatggctattctttcagcgtctcgta

SEQ ID NO: 32 - DNA, 64 bp, Homo sapiens
ggaggagtgtgaaggaattgatgggggcggggggtgaagcgacacscctttcaactcttcgtcg

SEQ ID NO: 33 - DNA, 63 bp, Homo sapiens
  misc_feature: wherein, v is g or c or a
gggagggtgggacggttgcggtggttggggtaaggcccaccscctttagccttcgvtttccct

SEQ ID NO: 34 - DNA, 64 bp, Homo sapiens
agaaggggggaccagggctagtgactgggcatgaagtgaaggacggtacattccaattggctgt

SEQ ID NO: 35 - DNA, 60 bp, Homo sapiens
atgatagaggtgggtgcgctccttgaggtggggagggggacgagtgtgacaccccggttg

SEQ ID NO: 36 - DNA, 64 bp, Homo sapiens
  misc_feature: wherein, s is g or c
atcgggattgtsgttccgagatgccattgatagtgaygatttynnvgrggtgaacccgctgtgg

SEQ ID NO: 37 - DNA, 62 bp, Homo sapiens
  misc_feature: wherein, n is g, a, t or c
ggnggcttggtccggtgaattctgtaagagttttattctagactcgcttgtttccttgccag